(19) United States
(12) Patent Application Publication
(10) Pub. No.: US 2002/0169407 A1
(43) Pub. Date: Nov. 14, 2002

(54) Support apparatus with interchangeable inserts

(76) Inventor: Brian T. Glinsboeckel, Kenmore, NY (US)

Correspondence Address: David L. Principe, Hodgson Russ LLP, Suite 2000, One M&T Plaza, Buffalo, NY, 14203-2391, US

(21) Appl. No.: 10/062,157

(22) Filed: Jan. 31, 2002

Related U.S. Application Data
(60) Provisional application No. 60/289,495, filed on May 8, 2001.

(51) Int. Cl. A61F 5/00

(52) U.S. Cl. 602/64; 602/20

(57) ABSTRACT

A support apparatus with interchangeable inserts includes a base member with an elongate body. The elongate body includes a hook and loop fastening surface for removably attaching interchangeable inserts. The base member attaches to the body of the user in end-to-end fashion. Securing straps and an anchoring ring provide additional support to keep the base member in position. The inserts are removably attached to the base member such that they are disposed against the body of the user when the apparatus is in use. The inserts may have different textures for tactile sensations and may be provided with cavities containing fluid or gel for transmitting heat or cold to the body of the user.

### FIELD OF THE INVENTION

The present invention relates generally to therapeutic support braces and more particularly relates to an improved construction and arrangement for a brace with interchangable inserts.

### BACKGROUND OF THE INVENTION

Prior art devices of this type have relied on a variety of different designs and constructions to support the wrist during repetitive actions such as typing, cumulative trauma, or sport related repetitive motions which impart injury, etc. Accordingly, a number of prior art devices have been developed that are made of reinforced synthetic fabric material, such as nylon that use multiple securing straps that are typically secured to oppositely disposed portions of the braces by VELCRO type hook and loop fasteners.

What is needed is a support apparatus that is capable of being used with an infinite number of interchangeable inserts for the application of heat, cold, or massage inside the support apparatus.

### SUMMARY OF THE INVENTION

The present invention meets the above described need by providing a support apparatus with interchangeable inserts that includes a base member with an elongate body. The elongate body includes a hook and loop fastening surface for removably attaching interchangeable inserts. The base member attaches to the body of the user in end-to-end fashion. Securing straps and an anchoring ring provide additional support to keep the base member in position. The inserts are removably attached to the base member such that they are disposed against the body of the user when the apparatus is in use. The inserts may have different textures for tactile sensations and may be provided with cavities containing fluid or gel for transmitting heat or cold to the body of the user.

### BRIEF DESCRIPTION OF THE DRAWINGS

The invention is illustrated in the drawings in which like reference characters designate the same or similar parts throughout the figures of which:

FIG. 1 is a perspective view of the base member of the present invention;

FIG. 2 is a perspective view of the base member of FIG. 1 in a curved position for wrapping around the arm of the user;

FIG. 3 is a perspective view of the base member of FIG. 1 in as it would appear after being wrapped around the user's arm and secured;

FIG. 4 is a perspective view of an alternate embodiment of the base member of FIG. 1;

FIG. 5 is a perspective view of the base member of FIG. 4 in a curved position for wrapping around the arm of the user

FIG. 6 is a perspective view of an insert for the base member;

FIG. 7 is a perspective view of the opposite side of the base member shown in FIG. 6;

FIG. 8 is a perspective view of an alternate embodiment of the insert;

FIG. 9 is a perspective view of another alternate embodiment of the insert;

FIG. 10 is a perspective view of the opposite side of the insert shown in FIG. 9;

FIG. 11 is a perspective view of another alternate embodiment of the insert;

FIG. 12 is a perspective view of another alternate embodiment of the insert;

FIG. 13 is a perspective view of the opposite side of the insert shown in FIG. 12;

FIG. 14 is a perspective view of another alternate embodiment of the insert;

FIG. 15 is a perspective view of a cover for the insert shown in FIG. 14; and,

FIG. 16 is a top plan view of an alternate embodiment of the present invention.

### DETAILED DESCRIPTION

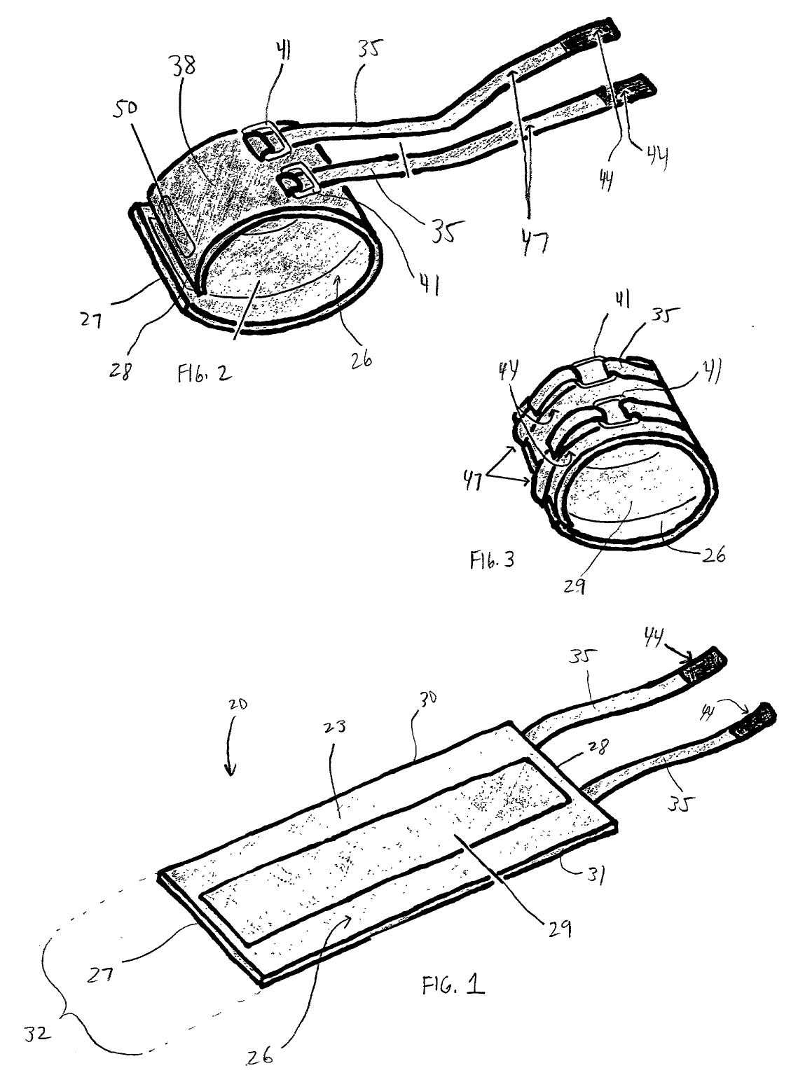

In FIG. 1, a base member 20 includes an elongate body 23 having a first surface 26. The body 23 extends in the longitudinal direction from a first end 27 to a second end 28. The body 23 is also bordered by a pair of opposed sides 30 and 31. The elongate body 23 may be constructed of any elastic material suitable for use as a support member. The material is stretchable with elastic characteristics and may also be water resistant. One material that is suitable for this purpose is sold under the brand name LYCRA. Other materials having similar characteristics would also be suitable for the material of the elongate body 23.

As shown in FIG. 2, the first surface 26 is located on the inside when the base member 20 is wrapped around the body of the user such as the wrist or forearm. Returning to FIG. 1, a hook and loop fastening surface 29 is disposed on the first surface 26 of body 23. As known to those of ordinary skill in the art, hook and loop fastening systems include a coacting hook portion on a first member and a loop portion on a second member. The hook and loop portions are capable of being removably attached to one another and provide a convenient fastening system that may be used in place of buttons or the like. A well known hook and loop fastening system is sold under the brand name VELCRO. Surface 29 may comprise either the hook or the loop portion of the hook and loop fastening system as will be described in greater detail below.

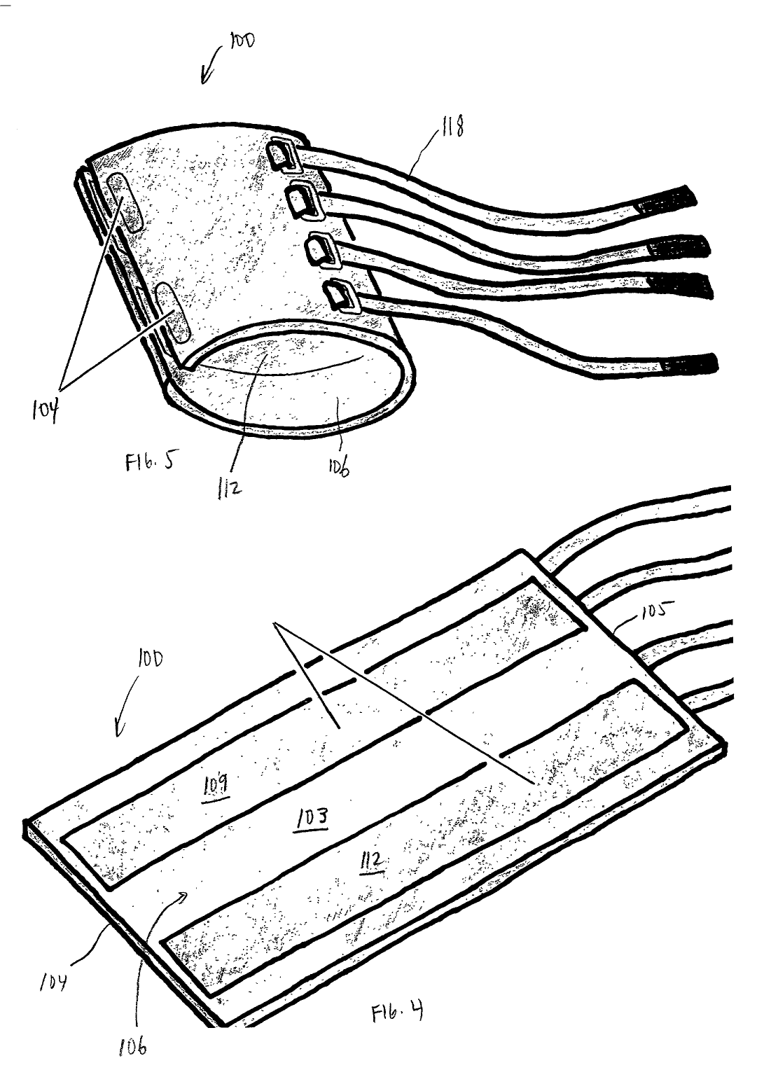

The body 23 has a major longitudinal dimension that varies depending on the intended use for the base member 20. The width 32 of the body 23 also varies depending on the intended use. For example, the body 23 shown in FIGS. 1-3 is intended for use as a wrist support, whereas the body 103 shown in FIGS. 4 and 5 is intended for use as a wrist and forearm support. Accordingly, the body 103 may be slightly longer and may be wider to accommodate the forearm of the user. Similar support members with varying dimensions may also be used for the hand, elbow, knee or foot. It is to be understood that the present invention may be applied to any support apparatus with interchangeable inserts and capable of being attached to the body of a user. The wrist and forearm mounted device shown in the illustrations is one example of the invention and the invention is not intended to be limited to a support apparatus mounted to the wrist or wrist and forearm.

In FIG. 2 a pair of straps 35 extend from a second surface 38 of the body 23. Although a pair of straps are shown, the number of straps is not critical and a single, wider strap or additional straps (FIGS. 4 and 5)could also be used. The second surface 38 is located on a side of the body 23 that is opposite from the first surface 26. When the base member 20 is wrapped around the wrist of the user, the second surface 38 faces outward. The straps 35 may be attached to the second surface 38 at a point between the ends 27 and 28 of body 23.

A pair of leverage rings 41 are attached to the second surface 38. The straps 35 have a first hook or loop portion 44 of a hook and loop fastening system disposed at the ends thereof and a second hook or loop portion 47 of a hook and loop fastening system disposed between the ends of the straps 35.

The second surface 38 also has a hook or loop portion 50 disposed near an end of the body 23. As shown, the opposing ends 27 and 28 are attached to one another in end-to-end fashion by means of hook or loop portion 50 and the hook and loop fastening surface 29 disposed on the first surface 26.

In FIG. 3, the straps 35 are shown as they attach around the body 23 to further secure base member 20 in position around the wrist of the user. The straps 35 are wrapped around the second surface 38 of body 23 and then inserted into the leverage rings 41. The straps 35 are folded about the leverage rings 41 and then the ends of the straps 35 extend in the opposite direction and the hook or loop portion 44 on the end of the straps 35 is attached to the hook or loop portion 47 disposed between the opposite ends of the straps 35.

Turning to FIGS. 4 and 5, in an alternate embodiment base member 100 has a body 103 that is suitable for use to support both the wrist and forearm. The body 103 has two opposed ends 104 and 105 and a first surface 106 with a pair of hook and loop fastening surfaces 109 and 112 disposed along the length of the body 103. As shown in FIG. 5, a second surface 115 of body 103 has a pair of hook and loop fastening surfaces 104 disposed at or near the end 105 of the body 103. The body 103 is attached around the wrist and forearm in end-to-end fashion and then the straps 118 are attached around the body 103 as described above in connection with FIGS. 1-3.

Turning to FIGS. 6-7, the hook and loop fastening surfaces 29, 102, and 112 are capable of receiving multiple inserts either individually or in combination as described below. Insert 200 has a first surface 201 containing a plurality of projections 203 that provide a tactile surface with massage-like qualities. A second surface 205 opposite the first surface has a hook and loop fastening surface 208 disposed thereon. The hook and loop fastening surface 208 is capable of removably attaching to surfaces 29, 109, 112 such that the insert 200 is held in position against the body of the user by the base member 20 or 100.

Turning to FIG. 8, in an alternate embodiment an insert 300 is formed out of a sheet of absorbent cotton or like material. The insert 300 provides a surface for absorbing the perspiration of the user.

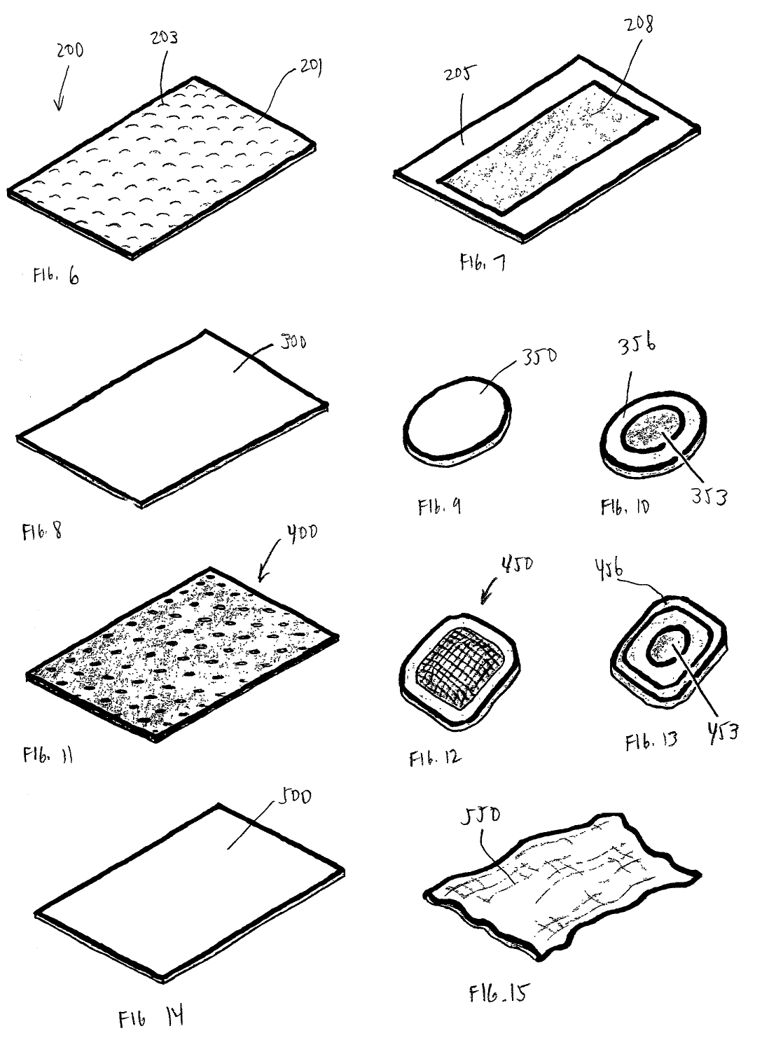

In FIGS. 9 and 10, an oval-shaped member 350 having a cavity is shown. Other shapes for the hollow member would also be suitable. A gel is disposed inside the cavity. A hook and loop fastening surface 353 is disposed on a bottom surface 356.

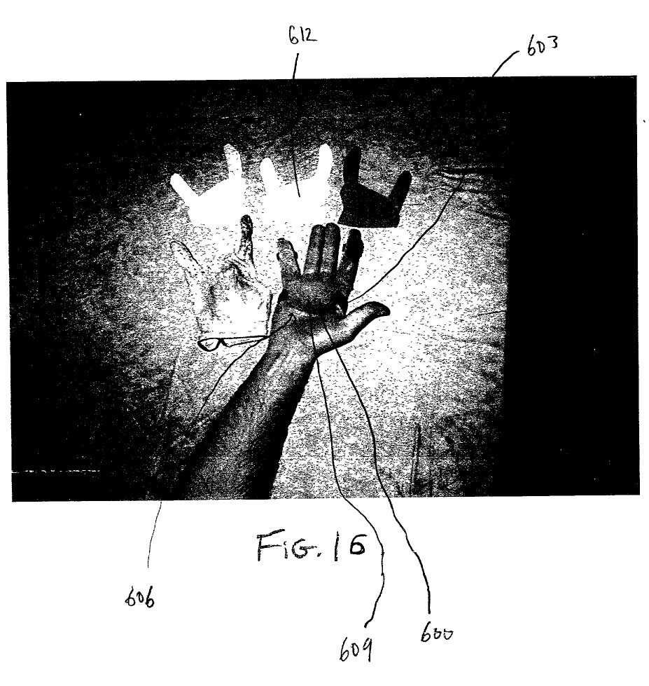

In FIG. 11, insert 400 is constructed of a perforated foam for ventilation. In FIGS. 12 and 13, an insert 450 has a magnet enmeshed in a member. A hook and loop fastening surface 453 is disposed on a bottom surface 456.

In FIG. 14, insert 500 comprises a gel insert that may be heated or cooled. In FIG. 15, a slip cover 550 is shown. The slip cover 550 provides for storing and protecting the insert between uses.

As described above, an infinite number of interchangeable inserts can be formed out of different materials for different purposes. The materials may have different textures for different tactile sensations. The materials may have internal cavities for storing different liquids that may be used for transmitting heat or cold to the body. The liquids may also be used to provide a different type of support than the solid surfaces.

Turning to FIG. 16, an alternate embodiment of the present invention is shown. A base member 600 is attached to the hand 601 of the user by straps 603 that may attach around the palm and around the fingers. The base member 600 includes a hook and loop fastening surface 606 disposed on the side opposite from first surface 609. A plurality of inserts 612 having different tactile surfaces or liquid or gel filled cavities, which can be similar to the inserts described above in connection with FIGS. 6-14, may be used interchangeably with base member 600. As described above, the inserts 612 are attached to the base member 600 by means of coacting hook and loop fastening surfaces.

While the invention has been described in connection with certain embodiments, it is not intended to limit the scope of the invention to the particular forms set forth, but, on the contrary, it is intended to cover such alternatives, modifications, and equivalents as may be included within the spirit and scope of the invention as defined by the appended claims.

What is claimed is:

1. A support apparatus with interchangeable inserts, the support apparatus, comprising:
an elongate body having a first side and a second side disposed opposite the first side, the first side having a hook and loop fastening surface disposed thereon, the elongate body having a first end and a second end opposite the first end, the first and second ends capable of being attached to each other so that the body can be disposed in end-to-end fashion;
at least one insert having a first side and a second side opposite the first side, the second side having a hook and loop fastening surface capable of cooperating with the hook and loop fastening surface on the elongate body.

2. The apparatus of claim 1, further comprising: at least one strap attached to the body at a first end of the strap and having a free second end disposed opposite the first end, the second end having a hook and loop fastening surface disposed thereon, the strap having a cooperating hook and loop fastening surface disposed between the first and second end.

3. The apparatus of claim 2, further comprising:
an anchoring ring attached to the body.

4. The apparatus of claim 1, wherein the first side of the insert has a plurality of projections.

5. The apparatus of claim 1, wherein the insert is hollow such that a cavity is formed therein.

6. The apparatus of claim 5, wherein a gel is disposed in the cavity.

7. The apparatus of claim 5, wherein a liquid is disposed in the cavity.

8. The apparatus of claim 5, wherein a solid material is disposed in the cavity.

9. The apparatus of claim 1, wherein the insert comprises a sheet of absorbent material.

10. The apparatus of claim 1, wherein the insert comprises a magnet.

11. The apparatus of claim 1, wherein the insert comprises a perforated material.

12. A support apparatus with interchangeable inserts, the support apparatus, comprising:
an elongate body having a first side and a second side disposed opposite the first side, the first side having a hook and loop fastening surface disposed thereon;
at least one strap attached to the body at a first end of the strap and having a free second end disposed opposite the first end, the second end having a hook and loop fastening surface disposed thereon, the strap having a cooperating hook and loop fastening surface disposed between the first and second end;
an anchoring ring attached to the body; and,
at least one insert having a first side and a second side opposite the first side, the second side having a hook and loop fastening surface capable of cooperating with the hook and loop fastening surface of the elongate body.

13. The apparatus of claim 12, wherein the first side of the insert has a plurality of projections.

14. The apparatus of claim 12, wherein the insert is hollow such that a cavity is formed therein.

15. The apparatus of claim 14, wherein a gel is disposed in the cavity.

16. The apparatus of claim 14, wherein a liquid is disposed in the cavity.

17. The apparatus of claim 14, wherein a solid material is disposed in the cavity.

18. A support apparatus with interchangeable inserts, the support apparatus comprising:
a base member shaped to conform to the hand of a user, the base member having a first side and a second side disposed opposite the first side, the first side having a hook and loop fastening surface disposed thereon;
at least one strap for attaching the base member to the hand of the user; and,
at least one insert having a first side and a second side opposite the first side, the second side having a hook and loop fastening surface capable of cooperating with the hook and loop fastening surface of the elongate body.

19. The apparatus of claim 18, wherein the first side of the insert has a plurality of projections.

20. The apparatus of claim 18, wherein the insert is hollow such that a cavity is formed therein.

21. The apparatus of claim 20, wherein a gel is disposed in the cavity.

22. The apparatus of claim 20, wherein a liquid is disposed in the cavity.

23. The apparatus of claim 20, wherein a solid material is disposed in the cavity.
